(19) United States
(12) Patent Application Publication
(10) Pub. No.: US 2002/0173549 A1
(43) Pub. Date: Nov. 21, 2002

(54) Compositions and methods for treatment of mild cognitive impairment

(76) Inventors: Richard J. Wurtman, Boston, MA (US); Robert K. K. Lee, Boston, MA (US)

Correspondence Address: Patent Administrator, KATTEN MUCHIN ZAVIS, Suite 1600, 525 West Monroe Street, Chicago, IL, 60661-3693, US

(21) Appl. No.: 09/986,470

(22) Filed: Nov. 8, 2001

Related U.S. Application Data
(60) Provisional application No. 60/246,615, filed on Nov. 8, 2000.

(51) Int. Cl. A61K 31/198; A61K 31/16; A61K 31/05

(52) U.S. Cl. 514/625; 514/565; 514/733

(57) ABSTRACT

A method of treating Mild Cognitive Impairment (MCI) has been discovered. The treatment includes administering an effective amount of a natural product that increases soluble amyloid precursor protein (APPs) expression. Examples of a natural product suitable for the method include, but are not limited to, resveratrol, capsaisin, olvanil, resiniferatoxin, arvanil, linvanil, capsazepine, or combinations of these naturally occurring substances. The treatment can also be used to prevent or alleviate the dementia, or to delay its onset. Moreover, a foodstuff is disclosed that incorporates a natural product useful in treating MCI.

### CLAIM OF PRIORITY

The present application is related to, and claims priority from, U.S. Provisional Application No. 60/246,615 filed Nov. 8, 2000, which is incorporated herein by reference in its entirety.

### STATEMENT OF FEDERAL SUPPORT

The present invention is made in whole or in part with financial support from the Federal Government under grant NIH #MH-28783. The Federal Government may have rights in the invention.

### FIELD OF THE INVENTION

The present invention relates to compositions and methods of treating Mild Cognitive Impairment (MCI) and related symptoms. MCI describes a set of non-disease symptoms, which in some cases may precede Alzheimer's Disease (AD). In fact, persons with MCI are at risk of developing AD. However, patients with MCI do not necessarily progress to full AD.

Currently there is no treatment to prevent or alleviate MCI or symptoms of MCI. There thus remains a need for an efficient and economic method and composition for treating or alleviating MCI.

### BACKGROUND OF THE INVENTION

Mild Cognitive Impairment is a condition that is considered to be within the range of normal function. Thus, MCI is not necessarily associated with excessive production of amyloidogenic precursor protein or formation of amyloid plaques. Yet those with MCI are at higher risk of developing AD and, likely, other neurodegenerative diseases. The term Mild Cognitive Impairment is synonymous with Age-Related Memory Impairment, Age-Consistent Memory Impairment, Late-Life Forgetfulness, Age-Related Cognitive Decline, and Relatively Inefficient Memory.

Although individuals with MCI generally have normal behavior and function, they have poor memory. Additional and more specific psychometric analysis is used to better define individuals with MCI.

One operational definition of MCI is a complaint of poor memory and objective evidence of memory performance that is one or more standard deviation below the mean for young adults. This definition uses as a standard the value for “restoration” of memory. Another psychometric definition of MCI is that the memory of the individual is one or more standard deviation below the mean for the individual's age group, a “parity” standard. Either the operational or psychometric definition is suitable to identify individuals suitable for treatment with the compositions of the invention.

A number of psychometric measures are useful to identify individuals with MCI. Memory can be tested by any of several methods known in the art, including learning and recalling of word lists, paragraphs and/or non-verbal materials. Other useful measures to determine whether an individual has MCI include, but are not limited to, the following: clinical dementia rating scale, clinical dementia rating sum of boxes, global deterioration scale, geriatric depression scale, mini-mental state examination, dementia rating scale, Wechsler Adult Intelligence scale, performance IQ, Boston naming test, controlled oral word association test, logical memory I test, logical memory II test, visual reproductions test one, visual reproductions test two, auditory verbal learning test-sum of learning trials 1 to 5, auditory verbal learning test I delayed recall/trial, the free and cued selective reminding test—sum of the performance across trials 1 to 6, and free and cued selective reminding test-delayed recall/trial 6×100. In general, individuals with MCI have psychometric scores that one or more standard deviation from the normal controls on one or more of the above measures. The controls can be age-and education-matched.

It is important to distinguish MCI from AD. In contrast to individuals with AD, individuals with MCI can have normal psychometric measures using the Wechsler adult intelligence scale—verbal IQ and the Wechsler adult intelligence scale—full-scale IQ.

By one preferred definition, individuals with MCI have general cognitive measures within 0.5 standard deviations of control subjects and also have memory performance 1.5 standard deviations below control subjects. An objective, documented decline in memory is useful in determining which individuals have MCI.

Individuals with clinically recognizable AD can have psychometric scores that are worse than individuals with MCI or normal controls. Among the psychometric tests useful to distinguish MCI and AD are: the clinical dementia rating scale—sum of boxes, the global deterioration scale, the mini-mental state examination, the dementia rating scale, the Wechsler adult intelligence scale—Verbal IQ, the Weschler adult intelligence scale—full scale IQ, the Wechsler adult intelligence scale—performance IQ, the Boston naming test, the Wechsler memory scale revised—logical memory I, the visual reproductions test one, the visual reproductions test two, the auditory verbal learning test—sum of learning trials 1-5, the auditory verbal learning test-delayed recall/trial 5×100, the free and cued selective remaining test—sum of the performance across trials 1 to 6, and the free and cued selective reminding test—delayed recall/trial 6×100. Individuals with MCI do not meet the criteria for AD. For example, those individuals with MCI can be distinguished from those with AD by a statistically significant difference on a panel of the above-listed psychometric tests.

### SUMMARY OF THE INVENTION

The present invention relates to a method of treating symptoms of Mild Cognitive Impairment (MCI) in a subject. The method comprises administering an effective amount of a composition comprising one or more naturally occurring substances that increase soluble amyloid precursor protein expression in the subject, wherein the increase in soluble amyloid precursor protein expression alleviates the symptoms of MCI. The substance is preferably a natural product present in plants, although other sources of natural products may be suitable.

The invention also includes a method of preventing or delaying the symptoms of MCI in a subject at risk thereof. The method comprises administering an effective amount of a composition comprising one or more substances that increase soluble amyloid precursor protein expression in the subject.

The substances in the composition may be natural products, including, but not limited to, resveratrol, capsaisin, olvanil, resiniferatoxin, arvanil, linvanil, capsazepine, anandamide, creatine, or combinations thereof. A preferred substance is resveratrol. The composition is preferably administered at least one time per day, to the subject being any animal, preferably a human, by various methods of administration known in the art, preferably orally. The natural product substances are effective at a wide range of concentrations, preferably at least about 0.1 mg to about 1000 mg/kg bodyweight per day. More preferred is a dose of at least about 1 mg to about 500 mg/kg bodyweight per day. Even more preferred is an effective amount of at least 10 mg to about 200 mg/kg bodyweight per day. Yet more preferred is a daily dose of 50 to 100 mg/kg body weight per day.

The substances in the composition may also include tryptophan and 5-hydroxytryptophan.

The invention also includes a method of increasing neuronal growth and/or synapse formation in a subject comprising administering to the subject an effective amount of a natural product, wherein neuronal growth or synapse formation is increased. The natural product should be administered at least one time per week to the subject, which can be a human, or an animal by various methods of administration known in the art, preferably orally. At least about 0.1 mg to about 1000 mg/kg body weight per day is needed, and preferably, at least about 1 mg to about 200 mg/kg body weight is used. Even more preferably a dose of about 50 to about 100 mg/kg body weight is used.

The invention also includes a method of treating the symptoms of a mild cognitive impairment in a subject comprising of administering a sufficient amount of dietary carbohydrate to increase brain tryptophan and serotonin levels.

Lastly, the invention also includes a foodstuff comprising a natural product and a carrier, wherein the natural product is resveratrol, capsaisin, olvanil, resiniferatoxin, arvanil, linvanil, capsazepine, anandamide, creatine, or combinations thereof, and wherein the carrier comprises a food, and wherein the foodstuff has the capacity to increase the expression of soluble amyloid precursor protein. Moreover, the foodstuff may contain other materials including food, fiber, salt, and flavors.

It is important to note that MCI is considered by those of ordinary skill in the relevant art to be a non-disease state. Hence, the subjects who would benefit the most from the present invention are those exhibiting symptoms of MCI but have not yet progressed to a clinical or manifest disease state, such as those subjects diagnosed as suffering from AD.

These and other objects of the invention will be evident to those of ordinary skill from a consideration of the discussions and descriptions provided in this specification, including the detailed description of the preferred embodiments.

It is to be understood that both the foregoing general description and the following detailed description are exemplary and are intended to provide further explanation of the invention claimed.

### BRIEF DESCRIPTION OF THE FIGURES

The accompanying drawings, which are incorporated in and constitute part of this specification, are included to illustrate and provide a further understanding of the methods and compositions of the invention. Together with the description, the drawings serves to explain the principles of the invention.

FIG. 1. Effect of Treatment with Resveratrol on Cellular APP Holoprotein and Secreted Soluble APP in Cultured Astrocytes.

FIG. 2. Effect of Anandamide, Methanandamide, 2-Arachidonyl glycerol, and Prostaglandin E2 on Levels of Amyloid Precursor Holoprotein.

FIG. 3. Effect of Varying Concentrations of Resveratrol on Total Abeta Media Concentration in CHO Cells Transfected with Human APP Gene Construct at Four Hours.

FIG. 4. Effect of WIN55212-2, a Cannabinoid Agonist, on Amyloid Precursor Holoprotein Synthesis in Response to Prostaglandin E2 in Cultured Astrocytes.

FIG. 5. Effect of Cannabinoids on Prostaglandin E2-Induced cAMP Increases in 10-Day Old Rat Cortical Neurons.

### DETAILED DESCRIPTION OF EMBODIMENTS OF THE INVENTION

Reference will now be made in detail to the present preferred embodiments of the invention, examples of which are illustrated in the accompanying drawings. The methods and compositions presented herein may be used to treat or prevent MCI.

Brief Glossary of Acronymns

APPh designates the holoprotein form of amyloid precursor protein.

2AG and 2-AG designate 2-arachidonyl glycerol, a lipid.

THC designates tetrahydrocannabinol.

Anan designates anandamide.

Methanan designates methanandamide.

PGE2 and PGE-2 designate prostaglandin E₂.

Compositions and Methods of the Invention

The inventors have identified methods of treating Mild Cognitive Impairment comprising: administering an effective amount of compositions that increase soluble amyloid precursor protein expression in a subject. The compositions include, for purposes of illustration and not limitation, non-steroidal anti-inflammatories, cannabinoids, vanilloids, or combinations of these agents. The compositions are natural products such as, for purposes of illustration and not limitation, resveratrol, capsaisin, olvanil, resiniferatoxin, arvanil, linvanil capsazepine, creatine, anandamide, marinol, or mixtures thereof.

“Effective amount” of an active compound, such as resveratrol or capsaisin, means a sufficient amount of the compound to treat, delay, alleviate, or prevent, the negative effects of Mild Cognitive Impairment at a reasonable benefit/risk ratio applicable. The specific effective dose level for any particular individual will depend upon a variety of factors including the stage of the condition being treated and the severity of the condition; activity of the specific compound employed; the specific composition employed; the age, body weight, general health, sex and diet of the subject; the time of administration, route of administration, and rate of excretion of the specific compound employed; the duration of the treatment; drugs used in combination or coinciding with the specific compound employed; and like factors well known in the medical and nutritional arts.

Natural product means a component of a plant, animal, fungus, or microorganism. The component can be a substance and in particular an isolated, or partially isolated substance, or it can be present in a crude mixture with other plant, animal, fungus, or microorganism components. An extract of the plant, animal, fungus, or microorganism can be prepared that is enriched in the natural product. Such an extract can be any extract known in the art, including an alcohol extract, a saline extract, an aqueous extract, or a hydrophobic solvent extract. The natural product can be further purified by methods known in the art, including chromatography, precipitation, extraction, and solvent partition.

The natural products suitable for use in the invention can increase secretion of soluble amyloid precursor protein. Soluble amyloid precursor protein has been shown to exhibit neuroprotective properties, increase neuronal outgrowth and regeneration, and promote synapse formation. Thus the invention enhances cognition, learning, and memory. Some of the natural products suitable for use in the invention can bind to cannabinoid receptors and transporters, and thereby regulate soluble APP (Amyloid Precursor Protein) secretion and cell-associated levels of APP holoprotein. The cannabinoid receptors and/or transporters can be coupled to cyclic AMP. By regulation of cyclic AMP, the agents can alter APP synthesis, APP expression, α-APP production, and soluble APP secretion.

Certain agents of the class of vanilloids, as disclosed herein, have neuroprotective properties. The agents may increase neuronal outgrowth, neural regeneration, or both. Furthermore, the agents may also promote synapse formation. Thus, treating with the agents of the invention can enhance cognition, learning, and memory. Examples of such agents include anandamide, an agonist at cannabinoid receptors, which has a chemical structure similar to the oleic acid homologue of capsaisin, and is known as olvanil. Capsaisin, a well-known natural vanilloid, is suitable for use in the invention. The vanilloids of the invention can regulate APP synthesis, APP expression, α-APP production, and soluble APP secretion. Agents effective at vanilloid receptors include, for purposes of illustrations and not limitation, capsaisin, resiniferatoxin, olvanil, arvanil, anandamide, linvanil, capsazepine, sesquiterpitol unsaturated dialdehydes, and triprenylphenols. These vanilloids, as well as others, are suitable for use in the invention, alone or in combination.

When alpha-secretase cleaves APP, soluble APP forms. Alternatively, beta-secretase and gamma-secretasecan also cleave APP to form Abeta. Unlike the hydrolysis site for beta secretase and gamma secretase, the hydrolysis site for alpha-secretase is within the Abeta region of APP; consequently, the hydrolysis of APP by alpha-secretase precludes formation of Abeta. Unlike Abeta, soluble APP has neuroprotective properties. It has now been discovered that use of natural NSAIDS, including resveratrol, cannabanoid agents, vanilloid agents, or combinations thereof, stimulate soluble APP formation. These agents can be useful for treating, delaying, and/or preventing the symptoms of Mild Cognitive Impairment.

What is more, it has been shown that certain substances, including resveratrol, agents active at cannabinoid receptors, and vanilloids are neuroprotective. In one aspect the substances enhance synapse formation and promote cognition and memory formation. These substances inhibit the abnormal increase in APP synthesis (mRNA and cell-associated protein) which is caused by elevations in cAMP levels. Thus, these substances can be used to prevent APP over-expression in brain cells. It has further been discovered that ion-gated channels can regulate APP overexpression. Action of the substances at ion-gated channels to regulate APP overexpression can contribute to the action of the substances. The substances of the invention can be formulated as a composition suitable for treatment of individuals.

The total daily dose of the compounds of the present invention administered to a subject in single or in divided doses can be in amounts, for example, from 0.0001 to 25 mg/kg body weight or more usually from 0.01 to 15 g/kg body weight. Multiple dose compositions may contain such amounts or submultiples thereof to make up the daily dose. In general, treatment regimens according to the present invention comprise administration to a human or other mammal in need of such treatment from about 1 mg to about 1000 mg of the active substance(s) of this invention per day in multiple doses or in a single dose of from 1 mg, 5 mg, 10 mg, 100 mg, 500 mg or 1000 mg.

In certain situations, it may be important to maintain a fairly high dose of the active agent in the blood stream of the subject, particularly early in the treatment. Hence, at least initially, it may be important to keep the dose relatively high and/or at a substantially constant level for a given period of time, preferably, at least about six or more hours, more preferably, at least about twelve or more hours and, most preferably, at least about twenty-four or more hours.

The compounds of the present invention may be administered alone or in combination or in concurrent therapy with other agents which affect the central or peripheral nervous system, particularly selected areas of the brain.

Liquid compositions for oral administration may include pharmaceutically acceptable emulsions, microemulsions, solutions, suspensions, syrups and elixirs containing inert diluents commonly used in the art, such as water, isotonic solutions, or saline. Such compositions may also comprise adjuvants, such as wetting agents; emulsifying and suspending agents; sweetening, flavoring, and perfuming agents. For example, the agents can be administered in flavored drinks, including fruit juice combination and chocolate-flavored milk-based drinks.

Solid compositions for oral administration may, for purposes of illustration and not limitation, include liquid, or chewable foodstuffs, capsules, tablets, pills, powders, gelcaps and granules. Suitable foodstuff forms include candy bars, granola bars, “power” bars, fruit bars, and milk-or-fruit-based drinks. In the solid dosage forms, the active compound may be admixed with at least one inert diluent such as a foodstuff, sucrose, lactose or starch. Such dosage forms may also comprise, as is normal practice, additional substances other than inert diluents, e.g., tableting lubricants and other tableting aids such as magnesium stearate and microcrystalline cellulose. In the case of capsules, tablets and pills, the dosage forms may also comprise buffering agents. Tablets and pills can additionally be prepared with enteric coatings and other release-controlling coatings.

Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings and other coatings well known in the pharmaceutical formulating art. The solid compositions may optionally contain opacifying agents and also be of a composition to release the active ingredient(s) only, or preferably, in a certain part of the intestinal tract, optionally in a delayed manner. Examples of embedding compositions, for illustration and not limitation, include polymeric substances and waxes.

Dosage forms for topical or transdermal administration of the inventive composition include, for illustration and not limitation, ointments, pastes, creams, lotions, gels, powders, solutions, sprays, inhalants or patches. The active component is admixed under sterile conditions with an acceptable carrier and any needed preservatives or buffers as may be required. Ophthalmic formulations, ear drops, eye ointments, powders and solutions are also contemplated as being within the scope of this invention.

In addition to the active compound of this invention, ointments, pastes, creams and gels may contain, excipients such as, for illustration and not limitation, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the active compounds of this invention, excipients such as, for illustration and not limitation, lactose, talc, silicic acid, aluminum hydroxide, calcium silicates and polyamide powder, or mixtures of these substances. Sprays can additionally contain customary propellants, such as chlorofluorohydrocarbons.

Transdermal patches have the added advantage of providing controlled delivery of active compound to the body. Such dosage forms can be made by dissolving or dispersing the compound in the proper medium. Absorption enhancers can also be used to increase the flux of the compound across the skin. The rate can be controlled by either providing a rate controlling membrane or by dispersing the compound in a polymer matrix or gel.

Accordingly, the present invention may treat or alleviate pre-disease states, especially MCI, transient ischemic accidents, hemorrhagic and occlusive stroke, and central or peripheral nervous system damage, dysfunction, or complications involving same stemming from edema, injury, or trauma. Such damage, dysfunction, or complications may be characterized by an apparent neurological, physiological, psychological, or behavioral aberrations, the symptoms of which can be reduced by the administration of an effective amount of the active compounds or substances of the present invention. In one embodiment, an effective amount of a composition comprising resveratrol is administered as treatment for occlusive stroke.

The natural products of the invention can be used in combination with neurotransmitter agonists, antagonists, and modulators. In a particular example, resveratrol can be administered to a subject having MCI, in combination with an effective amount of adenosine, adrenoreceptors, angiotensin, atrial naturiuretic peptide, bombesin, bradykinin, cholecystokinin, gastrin, dopamine, endothelin, GABA, glutamate, histamine, interleukin-1, serotonin, leukotriene, acetylcholine, carbachol, neuropeptide Y, nicotinic acetylcholine, opioid, platelet activating factor, prostanoid, purinoceptors, somatostatin, tachykinin, thrombin, vasopressin, oxytocin, vasoactive intestinal peptide, and the like. In a more particular example, the natural products of the invention such as, but not limited to, resveratrol, can be administered in combination with other memory-promoting agents, including, but not limited to, acetylcholinesterease inhibitors, CDP-choline, uridine, uridine/choline or neurotransmitter receptor agonists.

Further Aspects of the Embodiments

In one aspect, resveratrol is administered in an effective amount to effectively treat MCI and cerebral ischemia, and enhance synapse formation. Resveratrol inhibits prostaglandin formation, probably by inhibiting cyclooxygenase. Consequently, cyclic AMP levels in the brain decrease and the expression of APPs increase. In one embodiment of the invention, an effective amount of a resveratrol is administered to the subject suffering from MCI. The resveratrol may act as a non-steroidal antiinflammatory, as a neurotransmitter receptor antagonist, as a transport modulator, or a combination thereof, or by any other means. It is preferable that the resveratrol does not activate protein kinase C.

Capsaisin, another substance of the invention, effectively treats MCI and cerebral ischemia, and enhances synapse formation. Capsaisin binds to cannabinoid receptors and increases APPs.

Marinol, another substance of the invention, effectively treats MCI and cerebral ischemia, and enhances synapse formation. Marinol binds to cannabinoid receptors and increases APPs.

APPs has neuroprotective effects. APPs and agents that stimulate the formation of APPs increase neuronal outgrowth in cell culture, increase regeneration of neurons, and promote synapse formation. In consequence, or by other means, it is believed that APPs and agents that stimulate the formation of APPs enhance cognition, enhance learning, and enhance memory.

In contrast, cell-associated APP holoproteins also known as Abeta are associated with loss of cognition, poor memory, and AD.

In a particular embodiment, an effective amount of a modulator of a cannabinoid receptor, which is coupled to the cellular levels of cAMP, is administered to the subject suffering from MCI. As described herein, the modulator may comprise a receptor antagonist of a neurotransmitter, a cannabinoid transport modulator, or a combination thereof, preferably provided that the modulator does not activate protein kinase C.

In another embodiment, tryptophan and 5-hydroxy-tryptophan, are substances of the invention that effectively treat MCI and cerebral ischemia, and enhance synapse formation. Similarly, carbohydrates are substances of the invention that effectively treat MCI and cerebral ischemia, and enhance synapse formation.

The invention also encompasses a method of increasing the aromatic amino acid level, particularly brain tryptophan and serotonin levels in a subject comprising administering a sufficient amount of dietary carbohydrate. In mammals, the amino acid tryptophan is the precursor to serotonin synthesis in the brain. Certain carbohydrates when ingested can increase the ratio of trytophan to large neutral amino acids (T:LNAA) in the blood stream. This increase of T:LNAA allows a higher level of tryptophan to enter the brain, which is necessary for increasing serotonin synthesis. While carbohydrates from normal food can shift this T:LNAA ratio to a limited extent, these normal foods also contain fats and other ingredients like fibers and protein, some of which slow down digestion and otherwise interfere with the necessary shift in the balance of amino acids in the blood.

Because serotonin present in the bloodstream is excluded by the blood-brain barrier from entry into the brain, the administration of precursors such as L-tryptophan (L-TP) or L-5 hydroxytryptophan (L-5-HTP) is used to increase brain concentrations of serotonin (Wurtman and Fernstrom (1975) “Control of brain monoamine synthesis by diet and plasma amino acids.” The Ameican Journal of Clinical Nutrition, 28, 638-647), incorporated herein by reference in its entirety. The daily supplementation of precursors for serotonin comprises administering, for an effective daily period, an effective amount of L-tryptophan or preferably L-5-hydroxytryptophan as the intermediate precursors for serotonin (5-hydroxytryptamine). It is understood that any of its L, D or racemic forms are suitable, but preferably precursors are in L form. Furthermore, one skilled in the art will know to make tryptophan from 3-indolacetic acid or 3-indolpyruvic acid or use these acids as alternative to tryptophan and thus avoid the hepatic degradation by tryptophan pyrrolase. Other precursors or intermediates thereof are equally suitable with or without further modification, including but not limited to diethyl N-benzyloxycarbonyl-5-benzyloxycarbonyloxy-L-tryptophyl-L-aspartate, dibenzyl N-benzyloxycarbonyl-5-hydroxy-L-tryptophanylaspartate, 5-Hydroxy-L-tryptophyl-L-aspartic acid trihydrate, diethyl N-benzyloxycarbonyl-5-hydroxy-L-tryptophyl-L-glutamate, diethyl 5-hydroxy-L-tryptophyl-L-glutamate hydrochloride, dibenzyl L-benzyloxycarbonyl-5-hydroxytryptophyl-L-glutamate, 5hydroxy-L-tryptophyl-L-glutamic acid, pentachlorophenyl ester of N-benzyloxycarbonyl-5-hydroxy-L-tryptophan, methyl ester of N-benzyloxycarbonyl-5-hydroxy-L-tryptophyl-L-tyrosine, N-Acetyl-5-hydroxy-L-tryptophan, methyl ester of N-acetyl-5-hydroxy-L-tryptophyl-L-tyrosine, methyl ester of n-acetyl-5-hydroxy-L-tryptophyl-5-hydroxy-L-tryptophan, 5-hydroxy-L-tryptophyl-L-alanine hydrate, 5-hydroxy-L-tryptophan-L-valine, 5-hydroxy-L-tryptophyl-L-leucine, 5-hydroxy-L-tryptophyl-L-proline, 5-hydroxy-L-tryptophyl-L-phenylalanine, 5-hydroxy-L-tryptophyl-5-hydroxy-L-tryptophan, 5-hydroxy-L-tryptophyl-L-tryptophan, 1-5-hydroxytryptophyl-L-serine, 5-hydroxy-L-tryptophyl-L-arginine, 5-hydroxy-L-tryptophylglycine, 5-hydroxy 1-tryptophyl-gamma-aminobutyric acid, 5-hydroxy-L-tryptophanamide hydrate, methyl ester of 5-hydroxy-L-tryptophyl-L-histidine, benzyl ester of L-5 hydroxytryptophan, benzyl ester of N-benzyloxycarbonyl-5-hydroxy-L-tryptophyl-5-hydroxy-L-tryptophan, 5-Hydroxy-L-tryptophyl-5-hydroxy-L-tryptophan hemihydrate, 5-hydroxytryptophan inosinate, theophylline salt of (DL) 5-hydroxytryptophan, and the like.

These serotonin precursors can be administered alone or in combination with the stimulants of serotonin synthesis including but not limited to vitamin B1, vitamin B3, vitamin B6, biotin, S-adenosylmethionine, folic acid, ascorbic acid, magnesium, coenzyme Q10, and piracetam.

The substances of the invention are effective for preventing and treating MCI. In addition, the substances effectively enhance synapse formation and neuroprotection. Thus the substances may ameliorate the consequences of cerebral ischemia. Cerebral ischemia may be acute or chronic and includes transient ischemic accidents, head trauma, and stroke.

The present invention also encompasses a method of effectively preventing or delaying the manifestation of symptoms of MCI for subjects at risk comprising administering an effective amount of a natural product in which the natural product increases soluble amyloid precursor protein expression in the subject, in which the increase in soluble amyloid precursor protein expression prevents or delays the onset of the symptoms of MCI. In one aspect, the natural product suitable for use in the invention delays the onset of the symptoms of MCI.

Compositions of the Present Invention

Another embodiment of the invention encompasses compositions comprising a carrier and a natural product that modulate the expression, production, or formation of amyloid precursor protein (APPs) in a subject. The term “carrier” means a non-toxic, inert solid, semi-solid liquid filler, diluent, encapsulating material, formulation auxiliary of any type, or simply a sterile aqueous medium, such as saline. The carrier can include a sweetener. The foodstuff, comprising the modified natural product and carrier, has the capacity to increase expression of soluble amyloid precursor protein. In one aspect, the foodstuff delays or prevents the symptoms of a mild cognitive impairment in a subject. In another aspect, the foodstuff treats the symptons of MCI in a subject.

The carrier can be a food or a nutriceutical carrier. The carrier can be in the form of a powder for preparing a beverage, a lozenge or dissolving candy, a chewable food, a topping such as croutons or a salad dressing, or a prepared beverage. Among suitable carriers are fruit juices, beverages flavored with fruit in addition to or instead of other flavors, packaged food products including flavored or unflavored yogurt, candy bars, and granola or energy bars.

Some examples of suitable carriers are foodstuffs, sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances.

The following ingredients, according to the judgment of the formulator, wetting agents, emulsifiers and lubricants such as sodium lauryl sulfate and magnesium stearate, as well as coloring agents, releasing agents, coating agents, sweetening, flavoring and perfuming agents, preservatives and antioxidants may also be added to the composition. Examples of acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, alpha tocopherol and the like; and the metal chelating agents such as citric acid, ethylenediamine tetraacetic acid (EDTA), sorbitol, tartaric acid, phosphoric acid and the like.

### EXAMPLES

The following examples further illustrate, without limiting the present invention:

Experiments and exemplary procedures and formulations are described below which provide additional enabling support for the present invention. In particular, in vitro studies using primary cultures of rat cortical astrocytes and in vivo studies using appropriate animal models are disclosed.

General Methods

Astrocytes are isolated from cortices from postnatal rats by methods standard in the art. In brief, dissected cortices are dissociated by trypsinization and trituration through a flame-polished Pasteur pipette. Cells are plated onto poly-L-lysine coated 35- or 100 mm culture dishes at densities of about 10-25 cells/mm². The initial culture media, minimal essential medium (MEM, Gibco) containing 10% horse serum (BioWhittaker), are aspirated after 2-5 h after plating to remove unattached cells and debris, and replaced with MEM containing 7.5% fetal bovine serum (FBS, BioWhittaker). Half the media is replaced with MEM/7.5% FBS twice weekly. Cells are kept at 37° C. in a humidified 5% CO₂/95% air incubator. Media are changed twice weekly. Immunocytochemical staining with antibodies against GFAP and tau shows that >90% of cultured cells are astrocytes and <5% are neurons. Pharmacological manipulations are performed in serum-free media on 7-14 DIV confluent astrocytes.

Detection of Cell-Associated Protein

To detect cell-associated proteins (APP or GFAP), astrocytes from 35 mm dishes are scraped into lysis buffer (60 mm Tris/HCl, 4% SDS, 20% glycerol, 1 mm dithiothreitol), ultrasonicated and boiled for 5 mm. The total amount of cell protein per dish, estimated using the bicinchoninic acid assay, is not altered by pharmacological treatments. Bromphenol blue (0.1%) is added to each sample and equal amounts of protein (75 mg/lane) are loaded on 10% SDS polyacrylamide gels.

To detect secreted APP, culture media is collected after drug treatments and phenylmethylsulfonyl fluoride is added to a final concentration of 2 mM. The media samples are then applied to Sephadex PD-b desalting columns (Pharmacia) and eluted with distilled water. Column eluates are frozen and dried by vacuum centrifugation. The lyophilized proteins are reconstituted in 25 μL water followed by 25 μL of 2× Laemmli gel loading buffer, and boiled for 5 min.

The amount of media or cell protein loaded for sodium dodecyl sulfate-polyacrylamide gel electrophoresis (10-20% SDS PAGE; Bio-Rad) is normalized for the amount of protein per sample. Proteins (equivalent to 100 μg cell protein/lane) are separated by electrophoresis, electroblotted onto polyvinylidene difluoride membranes (Immobilon-P, Millipore) and blocked in Tris-buffered saline with 0.15% Tween 20 (TEST) containing 5% powdered milk for 30 min. After 2×10 mm rinses in TBST, the membranes are incubated in TEST containing an appropriate antibody. Monoclonal antibodies 22C11 and GFAP (both from Boehringer Mannheim) are used to detect the N-terminus of APP and glial fibrillary acidic protein respectively; antisera R37 and R98 (gifts of Dr. F. Kametani, Tokyo Institute of Psychiatry) are used to detected the C-terminus and KPI motifs of APP respectively; antiserum C8 (gift of Dr. D. Selkoe, Women's Hospital, Harvard Medical School, Cambridge, Mass.) is used to detect the C-terminus of APP.

After an overnight incubation, membranes are rinsed in TBST then treated for 1 h with a peroxidase-linked secondary antibody. After several rinses in TEST, protein bands are visualized on Kodak X-AR films by an enhanced chemiluminescence method (Amersham). Optical densities of the protein bands are quantitated by laser scanning densitometry (LICE, Bromma, Sweden), and normalized to the densities of those bands generated under control conditions.

cAMP Assay

Levels of cyclic AMP are measured with an [8-³H]-cAMP assay kit (Amersham TRK 432) in astrocytes grown on 35 mm dishes. In brief, after aspirating the medium and rinsing twice with 1 ml ice cold PBS, the cells are scraped in 0.8 mL ice cold ethanol and sonicated. The cell suspension is incubated for 5 nm at room temperature, centrifuged and the supernatant is dried in a rotary evaporator. After resuspension in 120 μL Tris/EDTA buffer, two duplicate samples of 50 μl each are mixed with the binding protein, [8-³H] adenosine 3′,5′-cyclic phosphate tracer and incubated at 2-4° C. for 2 h. A charcoal suspension (100 μL) is added to the samples before centrifugation and 200 μL of the supernatant is removed for scintillation counting. The amount of cyclic AMP (pmol/mg protein) is estimated by comparing to known standards, and normalized to the amounts of whole cell protein as determined by the bicinchoninic acid assay (Sigma).

Data Analysis

Measurements of cellular and secreted proteins, or of mRNA in treatment groups are normalized against those of control groups which are prepared in parallel and loaded onto the same blot. Analysis of variance (ANOVA) and t-tests are used to evaluate differences between groups (significance level, p=0.05), using drug treatments as the independent variable.

Analysis of RNA

Total RNA from astrocytes grown on 100 mm dishes is extracted by the acid guanidium thiocyanate-phenol chloroform method. In brief, the medium is aspirated and the cells are scraped in 1 mL of TRI Reagent. After incubation for 15 nm at room temperature, 0.2 mL chloroform is added, mixed vigorously with TRI Reagent and the mixture is stored for another 15 nm at room temperature. After centrifugation at 12,000 g for 15 mm, 0.5 mL isopropanol is added to the aqueous phase of the mixture to precipitate RNA. The RNA pellet collected by centrifugation (12,000 g, 15 nm at 4° C.) is washed with 70% ethanol once and solubilized in an appropriate amount of Formazol (Molecular Research Center, Cincinnati, Ohio). RNA samples (20 μg) are denatured by heating for 15 nm at 60° C. prior to loading onto 1-2% agarose-formaldehyde gels for electrophoresis. RNA is blotted onto Hybond polyvinyl membranes by overnight capillary transfer and fixed onto the membranes by UV light illumination. Membranes are pre-hybridized with Amersham Rapid-hyb (Amersham Lab, Arlington Heights, Ill.) buffer for 2 h and labeled overnight with a −p1.8 kb human APP cDNA or human glyceraldehyde-3-phosphate dehydrogenase probe (G3PDH; Clontech) labeled with [³²P]-dCTP using random primed extension (Amersham Megaprime DNA labelling kit). Membranes are dried and exposed to Kodak X-ray film for 24-48 h with an Amersham enhancer sheet. The relative amounts of mRNA obtained by hybridization are estimated using densitometric analysis of autoradiographs. The levels of APP mRNA are normalized to the amounts of G3PDH mRNA and expressed as a ratio to the levels of untreated, control cells.

Exposure of Astrocytes to cAMP, Resveratrol and Capsaisin

Confluent monolayers of astrocytes prepared according to Example 8.1 are treated with serum-free media containing 50, 100, or 250 μM 8Br-cAMP for varying durations (1 h, 6 h, 12 h, or 24 h). Levels of cAMP in astrocytes are measured with a [8-³H]-cAMP assay kit (Amersham TRK 432). Treatment, for 24 h, of astrocytes in culture with 1, 10 or 100 μM PG E₂ significantly increases the amounts of astrocytic APP mRNA (4.5 kb) relative to untreated cells. Similar increases in APP holoprotein (110-130 kD) are detected by mAb 22Cl1, antisera R37 or R98 on Western blots. APP secreted in the media (110-130 kD) is also increased by 24 h treatment with 1, 10 or 100 μM PG E₂using mAb 22Cl1, antiserum R37 or C8 immunodetection.

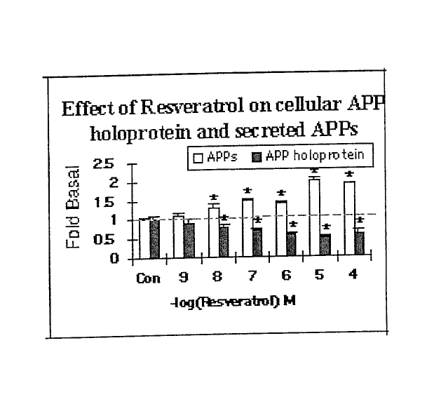

Referring now to FIG. 1, an average of four independent experiments, cultures of astrocytes are also treated for one hour with resveratrol (10⁻⁹ to 10⁻⁴M) and secretion of soluble APP (APPs) is measured by Western blot analysis. The dose response figure indicates that formation of soluble APP is stimulated about two-fold over control at 10 μM, and higher, resveratrol (p<0.05). Significant stimulation of APPs is also observed at 10 nM resveratrol and intermediate concentrations. Concomitant with the increase in APPs secretion is a decrease in the levels of cellular and amyloidogenic APP holoprotein in response to resveratrol. At 10 μM and higher resveratrol, the levels of amyloidogenic APP are decreased to about half (p<0.05) and a significant decrease in APP is observed at 10 μM resveratrol (p<0.05) and intermediate concentrations. The effects of capsaisin (10⁻⁹ to 10⁻⁶ M) are also observed.

Assays of PI hydrolysis are conducted as previously reported. Also, measurements of APPs are conducted by Western blot analysis.

Response of Amyloid Precursor Protein Levels to Anandamide

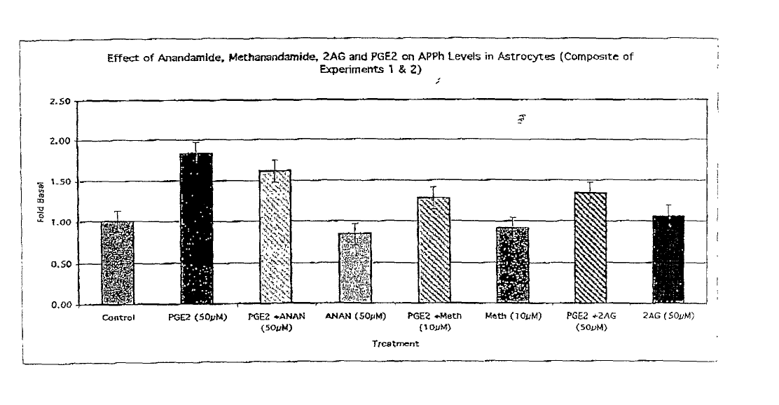

Referring now to FIG. 2, the change in amyloid precursor holoprotein levels is measured in cultured astrocytes. In two experiments, treatment of cultured astrocytes with 50 M prostaglandin E2 is found to result in about a 75% increase in APPh levels, compared to controls. Administration of 50 M anandamide is found to partially suppress the increase in APPh levels such that the increase is limited to about 55% over control. Anandamide (50 M) by itself is found to reduce APPh levels below control values. Methanandamide (10 M) is found to be more efficacious than anandamide (50 M) in its effect on PGE2-stimulated APPh levels, and effective at a lower concentration, i.e. more potent. 2-Arachidonyl glycerol (50 M) is found to not affect the basal levels of APPh, but is found to inhibit the PGE2-stimulated increase in APPh levels.

Resveratrol Dose Response of Total Abeta Levels

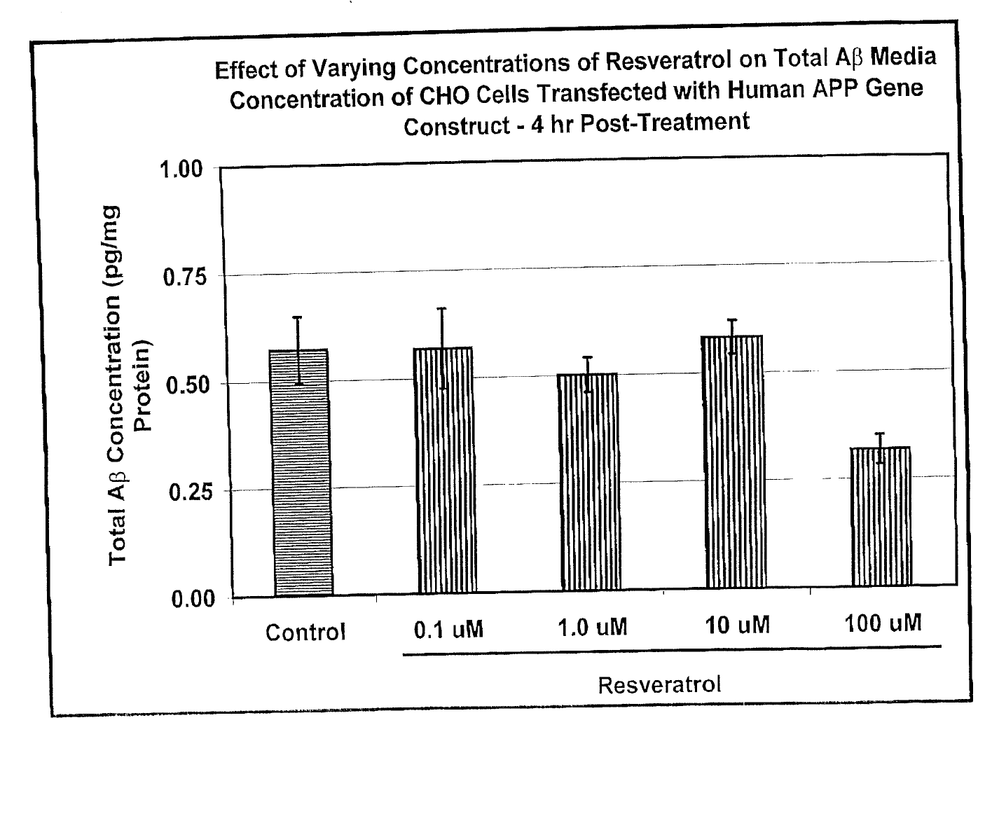

Referring now to FIG. 3, the change in Abeta levels in the media of Chinese Hamster Ovary (CHO) cells is measured in response to treatment with resveratrol at 0, 0.1, 1.0, 10, and 100 M. The Abeta levels are found to decrease to about half in response to a four-hour treatment with 100 M resveratrol, whereas lower concentrations are found not to differ from control.

Effect of a Cannabinoid Agonist on APPh Synthesis

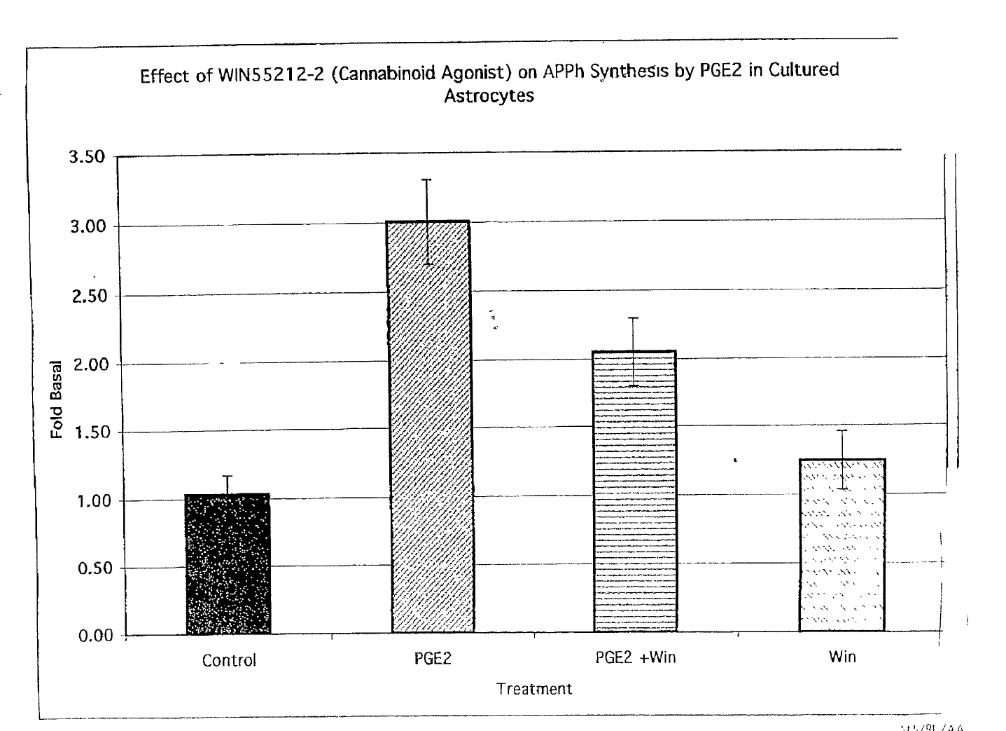

Referring now to FIG. 4, the effect of WIN55212-2 (100 M) on the synthesis of APPh stimulated by PGE2 in cultured astrocytes is measured. PGE2 is found to increase APPh levels three-fold and the addition of WIN55212-2 is found to inhibit the stimulation such that the increase in APPh levels is limited to two-fold. When administered in the absence of PGE2, Win55212-2 is found to have no statistically significant effect on APPh levels.

Effect of Cannabinoids of PGE2-Induced cAMP Increases in Ten-Day-Old Cortical Neurons

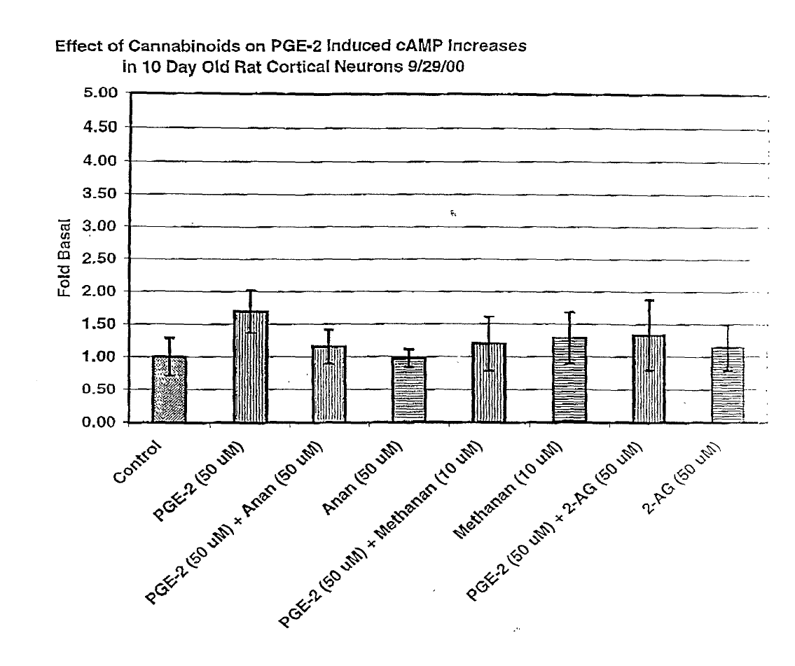

Referring now to FIG. 5, cell cultures of 10-day old cortical neurons are used to assess the affect of anandamide (50 M), methanandamide (10 M), and 2-AG (50 M) on PGE2-stimulated cAMP levels. PGE2 (50 M) is found to elevate cAMP levels about 1.6-fold over basal (control). Anandamide is found to suppress the PGE2-induced stimulation, but is found to have no effect on basal levels. Methanandamide, similarly, is found to inhibit the PGE2-induced increase in cAMP levels, but is found to have no effect on basal levels. 2-AG is found to likely have a similar inhibitory effect on stimulated cAMP levels without an effect on basal levels.

In vivo Studies

The present studies indicate that natural products with anti-inflammatory properties, such as resveratrol, can inhibit APP overexpression in GFAP-immunoreactive cultured astrocytes. Reactive astrocytes (that is, astrocytes that have been activated or stimulated in some fashion, e.g., those associated with brain or neuronal injury) in vivo also upregulate GFAP expression. The neuronal, brain, or head injury may give rise to the formation of reactive astrocytes, which overexpress APP and contribute to the formation of amyloid or neurotoxic APP derivatives.

Water Maze for Evaluation of Memory

Experimental Procedure

Spatial memory is assessed in a water maze, commonly termed a Morris water maze, or variations thereof. The nootropic effect of the natural products of the invention is assessed in the water maze. The rat's memory is evaluated by testing the rat's ability to find a submerged escape platform in a water maze using prior training and spatial clues from outside the maze. The rat's performance on the water maze test depends on its memory of the location of the submerged escape platforms, and its motor skills and exploration abilities.

The water maze is a round pool about 1.5 m in diameter with various large spatial cues on the walls of the room. The hidden escape platform is made of clear plastic with a friction surface and is submerged just under the water level. The water temperature is maintained at about 78° F. The room is uniformly and dimly lit.

Training for Search Behavior

To begin the training, the rat is placed in the maze to swim for up to one minute without the visual clues on the walls. After finding and climbing the platform, the rat is returned to its cage. The rats are trained three times per day for four days. The location of the submerged platform and the point of entry of the animal into the maze are changed for each training session. Thus, in the course of Morris water maze training, animals develop a search behavior. Animals with similar levels of search skills are used for further studies.

The trained animals are randomly assigned to an experimental or a control group.

Spatial Training

The spatial training is begun at three days after the training period. The tests continue for eight days with three trials each day. During the test period, the platform is placed in the same position in the pool, the rats are released at randomly chosen points, and the rats are allowed to swim for one minute.

The day after the end of spatial training, all rats are given a probe trial to assess their development of spatial bias. During the probe trial, the platform is removed from the pool and each rat is given 60 seconds of swimming.

Test for Memory Retention

After completion of the training phase, all the rats are kept for 35 days in their home cages with food and water ad libitum, but without experimental drugs. After that period, all animals are given a four-day retention test with three tests each day, in the same experimental room, with all spatial cues and platform position in the same location as in the search and spatial training. The handling procedure before and during the retention test is similar to the training procedures, but no drugs are given to the rats.

Data Measurement and Analysis

Swimming behavior is videotaped for analysis and archiving. Measurements are made of latency (time to search for the platform), the time spent in each sector of the pool, the mean swim path, swimming speed, float time (that is, at forward progress of less than 5 cm/s), and platform proximity (that is, change in spatial bias). Measurements are made by the methods standard in the art. The accuracy of the search is evaluated by a sector preference time and a site preference scalar, which counts instances of proximity to the presumptive platform. Data are analyzed by analysis of variance.

Experimental Observation

The effects of resveratrol on rat search behavior are addressed by analysis of rat search behavior and swimming ability after administering resveratrol. Four rats (about 24 months old) undergo spatial training for four days with three trials per day. Resveratrol is injected at 10 mg/kg body weight. Statistical comparisons are supplemented with anecdotal observations. After the last training trial, the rats are given a probe trial.

Thirty days after the probe trial, the rats are given a retention test, as above, in the absence and presence of resveratrol.

Induced Memory Impairment

Rats are injected with ibotenic acid into the medial septal area according to a standard model for impaired memory. Training in the maze is performed for three days, two weeks after treatment with ibotenic acid. The memory sparing effect of injection with resveratrol is evaluated using the methods indicated above.

Effect of Resveratrol on Delay of Onset of MCI

Two groups of subjects, group one given 2 mg dose of resveratrol twice daily and group two given 160 mg dose of resveratrol twice daily, are compared to an age, sex, and educational level-matched control group. All selected subjects are between the ages of 55 and 75. The control group receives a placebo. The subjects in the two experimental and control groups are evaluated on a twice-yearly basis with a battery of tests, consisting of two experimental control learning and recall of word lists to evaluate memory and the mini-mental state examination to evaluate dementia. The scores of the experimental and control groups are compared annually and followed for three years to evaluate delay in onset of MCI.

Effect of Resveratrol on Amelioration of MCI

Two groups of subjects, group one given 2 mg of resveratrol twice daily and group two given 160 mg of resveratrol twice daily, are compared to an age, sex, and educational level-matched control group. All selected subjects are between the ages of 55 and 75 and exhibit MCI based on a battery of tests. The control group receives a placebo. The subjects in the two experimental and control groups are evaluated on a twice-monthly basis with the same battery of tests, including learning and recall of word lists to evaluate memory and the mini-mental state examination to evaluate dementia. The scores of the experimental and control groups are compared monthly and followed for a year to evaluate memory.

Effect of Resveratrol on Prevention of MCI

Two groups of subjects, one given 2 mg dose of resveratrol twice daily and group two given 160 mg dose of resveratrol twice daily, are compared to an age, sex, and educational level-matched control group. All subjects selected are between the ages of 55 and 75. The control group receives a placebo. The subjects in the two experimental and control groups are evaluated on a twice-yearly basis with a battery of tests, consisting of learning and recall of word lists to evaluate memory and the mini-mental state examination to evaluate dementia. The scores of the experimental and control groups are compared annually and followed for three years to evaluate prevention of MCI by the doses of resveratrol.

Conclusion

Accordingly, the invention provides compositions and methods for preventing, alleviating, or delaying MCI by administering a composition comprising an agent or substance that stimulates levels of soluble APP.

It should be apparent to those of ordinary skill that other embodiments of the invention can be readily contemplated by those of ordinary skill in the art after reviewing the present specification and teachings. The present invention is not limited to the specific embodiments presented herein and should not be construed to exclude embodiments that fall within the scope and spirit of the invention

We claim:

1. A method of alleviating the symptoms of a mild cognitive impairment in a subject comprising administering an effective amount of a composition comprising one or more naturally occurring substances that increase soluble amyloid precursor protein expression in the subject.

2. The method of claim 1 wherein the substance comprises a natural product present in plants.

3. The method of claim 2 wherein the natural product is resveratrol, capsaisin, olvanil, resiniferatoxin, arvanil, linvanil, capsazepine, anandamide, creatine, or combinations thereof.

4. The method of claim 1 wherein the composition is administered at least one time per day.

5. The method of claim 1 wherein the subject is a human.

6. The method of claim 1 wherein the composition is administered orally.

7. The method of claim 1 wherein the effective amount is at least about 0.1 mg to about 1000 mg per kg body weight per day.

8. The method of claim 7 wherein the effective amount is at least about 10 mg to about 500 per kg body weight per day.

9. A method of preventing the symptoms of a mild cognitive impairment in a subject at risk of developing mild cognitive impairment comprising administering an effective amount of a composition comprising one or more naturally occurring substances that increase soluble amyloid precursor protein expression in the subject.

10. The method of claim 9 wherein the substance comprises a natural product present in plants.

11. The method of claim 10 wherein the natural product is resveratrol, capsaisin, olvanil, resiniferatoxin, arvanil, linvanil, capsazepine, anandamide, creatine, or combinations thereof.

12. The method of claim 9 wherein the composition is administered at least one time per day.

13. The method of claim 9 wherein the subject is a human.

14. The method of claim 9 wherein the composition is administered orally.

15. The method of claim 9 wherein the effective amount is at least 0.1 mg to about 1000 mg per kg body weight per day.

16. The method of claim 15 wherein the effective amount is at least 10 mg to about 500 mg per kg body weight per day.

17. A method of increasing neuronal growth or synapse formation in a subject comprising administering an effective amount of a composition that comprising one or more naturally occurring substances that increase soluble amyloid precursor protein expression in the subject.

18. The method of claim 17 wherein the substance comprises a natural product present in plants.

19. The method of claim 18 wherein the natural product is resveratrol, capsaisin, olvanil, resiniferatoxin, arvanil, linvanil, capsazepine, anandamide, creatine, or combinations thereof.

20. The method of claim 17 wherein the administering is at least one time per day.

21. The method of claim 17 wherein the subject is human or animal.

22. The method of claim 17 wherein the effective amount is at least 0.1 mg to about 1000 mg per kg body weight per day.

23. A method of treating the symptoms of a mild cognitive impairment in a subject comprising administering an effective amount of a composition wherein the composition comprises a compound selected from the group consisting of L-tryptophan, L-5-hydroxytryptophan, diethyl N-benzyloxycarbonyl-5-benzyloxycarbonyloxy-L-tryptophyl-L-aspartate, dibenzyl N-benzyloxycarbonyl-5-hydroxy-L-tryptophanylaspartate, 5-Hydroxy-L-tryptophyl-L-aspartic acid trihydrate, diethyl N-benzyloxycarbonyl-5-hydroxy-L-tryptophyl-L-glutamate, diethyl 5-hydroxy-L-tryptophyl-L-glutamate hydrochloride, dibenzyl L-benzyloxycarbonyl-5-hydroxytryptophyl-L-glutamate, 5-hydroxy-L-tryptophyl-L-glutamic acid, pentachlorophenyl ester of N-benzyloxycarbonyl-5-hydroxy-L-tryptophan, methyl ester of N-benzyloxycarbonyl-5-hydroxy-L-tryptophyl-L-tyrosine, N-Acetyl-5-hydroxy-L-tryptophan, methyl ester of N-acetyl-5-hydroxy-L-tryptophyl-L-tyrosine, methyl ester of n-acetyl-5-hydroxy-L-tryptophyl-5-hydroxy-L-tryptophan, 5-hydroxy-L-tryptophyl-L-alanine hydrate, 5-hydroxy-L-tryptophan-L-valine, 5-hydroxy-L-tryptophyl-L-leucine, 5-hydroxy-L-tryptophyl-L-proline, 5-hydroxy-L-tryptophyl-L-phenylalanine, 5-hydroxy-L-tryptophyl-5-hydroxy-L-tryptophan, 5-hydroxy-L-tryptphyl-L-tryptophan, 1-5-hydroxytryptophyl-L-serine, 5-hydroxy-L-tryptophyl-L-arginine, 5-hydroxy-L-tryptophylglycine, 5-hydroxy 1-tryptophyl-gamma aminobutyric acid, 5-hydroxy-L-tryptophanamide hydrate, methyl ester of 5-hydroxy L-tryptophyl-L-histidine, benzyl ester of L-5-hydroxytryptophan, benzyl ester of N-benzyloxycarbonyl-5-hydroxy-L-tryptophyl-5-hydroxy-L-tryptophan, 5-Hydroxy-L-tryptophyl-5-hydroxy-L-tryptophan hemihydrate, 5-hydroxytryptophan inosinate, theophylline salt of (DL) 5-hydroxytryptophan, and pharmaceutically acceptable salts thereof.

24. A method of treating the symptoms of a mild cognitive impairement in a subject comprising admisintering a sufficient a,ountof dietary carbihydrate to increase brain tryptophan and serotonin levels.

25. A method of treating the symptoms of occlusive stroke in a subject comprising administering an effective amount of a composition comprising one or more naturally occurring substances that increase soluble amyloid precursor protein expression in the subject.

26. The method of claim 25 wherein the composition comprises a compound selected from the group consisitng of resveratrol, capsaisin, olvanil, resiniferatoxin, arvanil, linvanil, capsazepine, anandamide, creatine, and combinations thereof.

27. A foodstuff comprising a natural product and a carrier, wherein the natural product is resveratrol, capsaisin, olvanil, resiniferatoxin, arvanil, linvanil, capsazepine, anandamide, creatine, or combinations thereof and the carrier comprises a food.
